(19) United States
(12) Patent Application Publication
(10) Pub. No.: US 2011/0104791 A1
(43) Pub. Date: May 5, 2011

(54) Media and Process for Culturing Algae

(75) Inventor: Randhir Singh Gajraj, Mumbai (IN)

(73) Assignee: INNOVATIVE CREATIONS BUSINESS MODULES PVT. LTD., Mumbai, MH (IN)

(21) Appl. No.: 13/000,513

(22) PCT Filed: Jun. 16, 2009
(86) PCT No.: PCT/IN09/00362

§ 371 Date: Dec. 21, 2010

(30) Foreign Application Priority Data
Jun. 24, 2008 (IN) 1323/MUM/2008

(51) Int. Cl. C12N 1/12

(52) U.S. Cl. 435/257.1

(57) ABSTRACT

The present invention is directed towards curtailing the use of harsh saline atmosphere and producing algal biomass free from salinity. It provides a salinity free medium and a process for culturing and producing algal biomass preferably Dunaliella biomass free from salinity and rich in natural mixed carotenoid content.

### FIELD OF INVENTION

The present invention relates to a salinity free medium and a process for culturing algae. More particularly the present invention provides a salinity free medium and process for culturing and producing Dunaliella biomass rich in natural mixed carotenoid content.

### BACKGROUND OF INVENTION

Dunaliella alga are motile, unicellular green algae belonging to the class Chlorophyceae. They are commonly found in marine water. Variety of species of dunaliella has been reported of which Dunaliella Salina is a typical halophilic micro alga with reddish brown color and found in brine. Only quite few organisms can survive in such highly saline conditions as salt evaporation ponds with salinity as high as 32% or even more. To survive, these organisms accumulate very high concentrations of β-carotene embedded in Cis oil or lipid fractions in the outer membranes of the cell to protect especially the DNA and other cell organelles against the intense light with quite harmful and mutation causing UV-A and UV-B radiations. Dunaliella Salina is rich source of natural mixed carotenoids such as beta-carotene, alpha-carotene, cryptoxanthin, zeaxanthin, lutein and lycopene, other antioxidants and vitamins. It is used in dietary, colouring, cosmetics and pharmaceutical applications. The said alga also accumulates high concentrations of osmotic balancing internal glycerol to provide protection against osmotic pressure induced by high salt concentrations in marine atmospheres. This offers an opportunity for commercial biological production of these substances.

The commercial cultivation of Dunaliella for the production of β-carotene throughout the world is now one of the success stories of halophilic or marine biotechnology. Different technologies are used, from low-tech extensive cultivation in and around salt lagoons to intensive cultivation at high cell densities under carefully controlled conditions in many countries like USA, Israel, Australia and India. Presently, the production or culturing of Dunaliella typically requires saline atmosphere along with the use of sodium chloride and other inorganic salts and their derivatives to create an osmotic balancing atmosphere/system much needed for alga Dunaliella species to survive and grow especially for production and processing of organic Natural Mixed Carotenoids.

JP 2003325165 discloses method for culturing and producing the green algae Dunaliella in concentrated seawater.

U.S. Pat. No. 4,554,390 discloses a method for harvesting algae of the genus Dunaliella from suspensions thereof in brines containing sodium chloride at a concentration of about 3M or above, wherein the algal suspension is contacted with an adsorbent having a hydrophobic surface so as to adsorb the algae thereon, and the adsorbent with the algae adsorbed thereon is separated from the brine. Beta-carotene and other useful cell components may be extracted from the adsorbed algae by treatment with a suitable solvent.

U.S. Pat. No. 4,115,949 discloses a process for the production of glycerol and proteinous substances of nutritive value which comprises cultivating algae species of the Dunaliella genus in a nutrient medium containing the mineral requirements of growth of the algae, said nutrient medium having a sodium chloride content of at least 1.5 M, the cultivation being effected while an adequate supply of carbon dioxide is provided and continued until a maximum concentration of algae is obtained, and continuing the cultivation of the algae in a nutrient medium having a content of sodium chloride of at least 3 M, cultivating the algae in this second nutrient medium until a high glycerol content is established, harvesting the algae, recovering from same the glycerol, and recovering the residue having a high protein content. The nutrient medium contains 1 mM to 10 mM Mg@++, 1 mM to 10 mM K@+, 0.1 mM to 20 mM Ca@++, Fe-EDTA about 0.5 .mu.m to 45 .mu.m, SO4@=about 1 mM to 5 mM; and NO3 about 1 mM to 20 mM; PO4@=about 0.01 mM to 1 mM.

U.S. Pat. No. 6,936,459 relates to a novel medium and a process using the novel medium for producing betacarotene and other carotenoids. The medium comprises a salt solution complex in the specified proportion starting from fresh water for the production and maximization of betacarotene and its isomers using an unique strain (ARL5) of the algae Dunaliella salina in a single stage of active growth. The process comprises of culturing Dunaliella salina ARL5 (CCAP 19/36) to produce algae cells having betacarotene and its isomers as well as a a high protein biomass by (a) growing Dunaliella salina ARL5 in an aqueous medium comprising: a salt solution complex comprising KCl, MgSO4 and NaCl wherein the salt solution complex comprises 0.2M-1M KCl, 0.41M-1.5M MgSO4, 1M-5M NaCl; and (b) recovering carotenes from said algae cells.

The invention also relates to a new strain of Dunaliella Salina ARL5 tolerates KCl at a concentration of upto 1M. This patent discloses culturing algae in a saline atmosphere.

As Dunaliella is known to survive or grow in a saline atmosphere, the current cultivation of Dunaliella is carried out at a high concentration of sodium chloride that is in saline atmosphere. The media and process employed currently are complex, cumbersome, capital intensive, require high energy inputs, quite harsh weather conditions and other biotic factors and habitats. The scorching sunlight with powerful mutation inducing UV-A and UV-B rays further may have adverse impact on the characteristics, growth and productivity of the algae. Such harmful conditions may be detrimental to the health of the people working in the area and also to the various plant and machineries involved in the entire processes. Moreover, for the consumption of salt rich Dunaliella, various processes have to be employed to make it free from salinity, which again is quite cumbersome. Further, the disposal of the spent medium left after culturing Dunaliella having very high concentrations of sodium chloride and other minerals as well as organic biomass left after processing may create ecological imbalance and lead to environmental pollution.

### A BRIEF DESCRIPTION OF THE INVENTION

It is an object of the present invention to curtail the use of saline atmosphere and to provide algal biomass free from salinity.

It is an object of the present invention to curtail the use of saline atmosphere and provide a Dunaliella biomass free from salinity.

It is an object of the present invention to provide a salinity free media for culturing and producing algal biomass.

It is an object of the present invention to provide a salinity free media for culturing and producing Dunaliella biomass.

It is an object of the present invention to provide a salinity free media for culturing and producing Dunaliella biomass rich in natural mixed carotenoid content.

Accordingly, in one of the aspect the present invention provides a salinity free media comprising at least one osmotically active substance.

Another object of the present invention is to provide a process for culturing and producing Dunaliella biomass rich in natural mixed carotenoid content.

Accordingly, in one of the aspect the present invention provides a process for culturing and producing Dunaliella biomass rich in natural mixed carotenoid content comprising: culturing an algal in the said salinity free medium, allowing the culture to grow under suitable cultural conditions and harvesting the Dunaliella biomass rich in natural mixed carotenoid content.

It is also an object of the present invention to provide a culture of Dunaliella rich in natural mixed carotenoid content and having high temperature and pH tolerance range.

It is yet another object of the present invention to provide a Dunaliella biomass rich in natural mixed carotenoid content, free from salinity and having palatable taste,

### DETAILED DESCRIPTION OF THE INVENTION

The present invention is directed towards curtailing the use of harsh saline atmosphere and producing algal biomass free from salinity. The present invention provides a salinity free medium and process for culturing and producing algal biomass. In preferred embodiments, the present invention provides a salinity free medium and process for culturing and producing Dunaliella biomass rich in mixed carotenoid content.

The term “salinity free medium” as used in the present invention refers to the medium comprising sodium chloride not exceeding 5 g/l that is having salinity much lesser as compared to the salinity of the sea water comprising sodium chloride in the range of 30-38 g/l.

According to an embodiment of the present invention, a salinity free media is provided comprising at least one osmotically active substance or osmolyte producing Dunaliella biomass rich in natural mixed carotenoid content. The said media can be used for culturing, growing, or maintaining the Dunaliella biomass.

The osmotically active substance or osmolyte used in the medium is selected from but not limited to sugar alcohol, sugar, sucralose, palm sugary sap or combinations thereof. The osmotically active substance is provided at such a concentration so as to sustain the growth and survival of the algae in osmotically balanced environment.

The sugar alcohol may be used in the concentration of from about 30 g/l to about 350 g/l. The sugar may be used in the concentration of from about 40 g/l to about 450 g/l. The sucralose may be used in the concentration of from about 40 g/l to about 450 g/l. The palm sugary sap when used it may be in the concentration of atleast double strength. In one of the preferred embodiment the concentration of palm sugary sap is not more than triple strength. The sugar alcohol and sugar when used in combination the total concentration may be upto 30 g/l.

In one embodiment of the present invention the sugar may be sucrose.

In an embodiment of the present invention the palm sugary sap may be obtained from palm tree. In a preferred embodiment the palm sugary sap is obtained from Palmyra palm. The palm sugary sap is commonly known as Neera in India.

In one embodiment of the present invention the sugar alcohol may be glycerol.

In an embodiment of the present invention the salinity free medium further comprises minerals from organic or inorganic source, and at least 0.1% of NaCl.

The minerals added in the salinity free media of the present invention may be selected from but not limited to carbonates or bicarbonates, nitrates, phosphates, sulphates, chlorides, magnesium, calcium, iron, boron, NPK complex, trace elements and or combination thereof.

The carbonates or bicarbonates may be selected from but not limiting to carbonates or bicarbonates of sodium, potassium, magnesium and/or combination thereof present in the range from about 0.01 g/l to about 1 g/l. The nitrates may be selected from but not limiting to nitrates of sodium, potassium, calcium and/or combination thereof, present in the range of about 0.00125 g/l to about 0.125 g/l. The phosphates may be selected from but not limiting to phosphate of mono or di forms of potassium, sodium and/or combination thereof, present in the range of about 0.005 g/l to about 0.1 g/l. The sulphates may be selected from but not limiting to sulphates of magnesium, calcium, iron, sodium, potassium and/or combination thereof, present in the range of from about 0.00009 g/l to about 0.65 g/l, The chlorides may be selected from but not limiting to calcium, magnesium, iron and/or combination thereof, present in the range from about 0.00014 g/l to about 0.14 g/l, The boron may be added in the form of boric acid, present in the range from about 0.00006 g/l to about 0.006 g/l. The NPK complex may be present in the range from about 0.001 g/l to about 0.15 g/l. The trace element may be selected from but not limiting to including copper, zinc, cobalt, manganese, molybdenum and/or combination thereof, present in ppm levels.

In the preferred embodiment of the present invention the salinity free medium comprises sodium chloride in the range of about 1 g/l to about 2 g/l.

According to yet another embodiment of the present invention the salinity free medium optionally comprises vitamins, extract from blue green algae and/or combination thereof.

The vitamins added in the in the salinity free media of the present invention may be vitamin B complex.

The blue green algae extract for use in the present invention medium may be prepared by adding blue green algae biomass in normal potable water and kept for atleast 4 hours, the supernatant was separated and used in the medium. Optionally, the blue green algae extract may be prepared by adding blue green algae biomass in 0.05 M phosphate buffer or in 0.1 M calcium chloride solution and kept for atleast 2 hours, the supernatant was separated and used in the medium.

The salinity free media of the present invention is prepared in fresh water that is potable water.

According to another embodiment of the present invention the salinity free media after culturing of the Dunaliella may be employed in the production of alcohol preferably ethanol and 1, 3-propanediol (PDO) having immense application in the industry. For such production of alcohol yeast or bacteria may be added to the media.

According to still another embodiment of the present invention, a process is provided for producing Dunaliella biomass rich in natural mixed carotenoid content. The process comprises the steps of: culturing Dunaliella in the salinity free media comprising at least one osmotically active substance; allowing the culture to grow under suitable cultural conditions and harvesting the Dunaliella biomass rich in natural mixed carotenoid content.

The species of Dunaliella used for the purpose of the present invention may be selected from Dunaliella salina and Dunaliella bardawil. In one of the preferred embodiment the Dunaliella species used is Dunaliella salina. In one of the most preferred embodiment the Dunaliella salina used is isolated from Sambhar lake in Ajmer district of Rajasthan, India. The same has been designated as Dunaliella salina SLS1 and was deposited with the Culture Collection of Algae and Protozoa at SAMS Research Marine Laboratory, Dunbeg, Argyll, PA371QA, UK under the Budapest Treaty. The same was provided with an accession number CCAP 19/37 on Jun. 13, 2008.

However, other halophilic species of algae, growing in the saline conditions may also be used in the present invention.

In one of the embodiment, for culturing the Dunaliella salina as per the present invention a seed culture or inoculum may be prepared by culturing the isolated Dunaliella saline in a modified ATCC:1174 DA medium. The pH may be maintained in the range of 5.8-10.8.The temperature may be preferably between 18⁹C to 489.C. The light intensity maintained may be preferably between 10-120 klux.

The salinity free medium comprising at least one osmotically active substance and minerals as described herein above is used in the process of the present invention.

The suitable cultural conditions under which the culture may be grown to obtain the Dunaliella biomass rich in natural mixed carotenoid content include pH, temperature, light intensity and/or growth period.

As per the present invention, the pH of the Dunaliella culture may be maintained between the range of 5.8 to 10.8. In the preferred embodiments, the pH of the culture may be maintained in the range of 8.6-9.4,

The temperature of the Dunaliella culture may be maintained between 2° C. to 60° C. In the preferred embodiments, the temperature of the culture may be maintained between 20° C. to 42° C.

The light intensity may be maintained between 5-120 klux. In the preferred embodiments, the light intensity may be maintained between 40-80 klux.

The Dunaliella culture may be allowed to grow for a period of atleast 10 days. In the preferred embodiments, the Dunaliella culture may be allowed to grow for a period of 12 to 14 days.

The Dunaliella salina SLS1 alga cultured as per the process of the present invention is atleast 2 times more active in terms of its average motility than the conventional strain of Dunaliella. The cultured Dunaliella salina SLS1 is microscopic, 6-12 micron in dimension and is smaller compared to the conventional strain of Dunaliella salina having dimension of 9-16 micron. The cultured Dunaliella salina SLS1 is round to oval in shape as compared to oval to spindle shaped conventional strain of Dunaliella salina.

Dunaliella salina SLS1 flagella are slightly longer than conventional strain of Dunaliella, and have more whiplash movements.

The algae cultured as per the present invention, has a temperature tolerance limit of −2° C. to 60° C. It has a pH tolerance range of 5.8 to 10.8.

The Dunaliella biomass rich in natural mixed carotenoid content may be obtained by harvesting the cultured Dunaliella salina by a suitable technique, preferably including centrifugation, flocculation with alum or ferric chloride to provide a wet algal biomass.

The wet algal biomass as obtained above may be further subjected to a suitable drying technique selected from but not limiting to treatment with alcohol, spray drying or lyophilization to obtain Dunaliella biomass in the dry form.

The Dunaliella biomass yield obtained on dry basis ranges from about 80 mg/l of the media per day to about 100 mg/l of the media per day.

The Dunaliella biomass thus obtained is free from salinity and has palatable taste.

The Dunaliella biomass as obtained by the process of the present invention is rich in natural mixed carotenoid content, vitamins, amino acids, glycerol and other active ingredients.

The Dunaliella biomass as obtained by the process of the present invention may be analyzed by standard UV spectrophotometric method at 450 nm to determine the content of mixed carotenoids. The individual carotenoids such as trans beta carotene, cis beta carotene, alpha carotene and others may be determined by HPLC method employing Waters make HPLC series 2487.

The Dunaliella biomass as obtained by the process of the present invention comprises higher concentration of natural mixed carotenoids, which may be as high as 12%-20%. The natural mixed carotenoids comprised in the Dunaliella biomass are composed of trans beta carotene in the range of about 12% to about 15%, cis beta carotene in the range of about 1.5% to about 1.8%, alpha carotene in the range of about 0.8% to about 1% and other carotenoids.

The Dunaliella biomass as obtained by the process of the present invention having better organoleptic characteristics either in wet or dry form can be used or after processing it further may be used as or in various health supplements, food, beverages, feed, agricultural, cosmetics, colouring and pharmaceuticals.

The Dunaliella biomass as obtained by the process of the present invention may be processed further with vegetable oils, solvents or by super critical extraction with carbon dioxide for extraction of natural mixed carotenoids.

The natural mixed carotenoids thus extracted may be used. as an antioxidant, therapeutic, chemopreventive, cosmetics, colouring and various other applications.

The present invention is directed towards curtailing the use of harsh saline atmosphere and producing algal biomass free from salinity. Since the algal biomass obtained is free from salinity, it is palatable has better organoleptic characteristisc and therefore may be used better suitability for various nutraceutical, food, feed, pharmaceutical and other such applications. Further, on account of being free from salinity, the algal biomass does not require further processing steps, chemicals, large amount of water, equipments and manpower. Thus, the process as per the present invention is simpler, less labour and cost intensive. Further, the invention incorporates a dual use of the salinity free medium not only for producing algal biomass with high content of natural mixed carotenoids but also in the production of ethanol and bio PDO so as to avoid the wastage of the media after the culturing of algae in order to curtail environmental pollution hazards.

The invention is further illustrated in the following non-limiting examples.

### EXAMPLES

### Example 1

Isolation of Dunaliella Salina SLS1 and Preparation of Inoculum:

Dunaliella salina SLS1 was isolated from salt pans of Sambhar lake by serial dilution and purification techniques in normal brine solution. To prepare the inoculum Dunaliella salina SLS1 was grown in test tubes and flasks under lab condition at pH 7.8-9.8, temperature 24-28° C., light intensity of 10 Klux in the ATCC: 1174 DA medium. Once grown in an exponential phase they were transferred in medium of 1-2 Molar NaCl strength in potable water with inputs of minerals as depicted below in Table 1 to further grow them in plastic buckets and use them as inoculum to carry out further growth and natural mixed carotenoid accumulation experiments with different osmolytes likes of Sugar alcohol, Sugar and Neera.

TABLE 1

| Mineral salts | Concentration g/l |
|---|---|
| Sodium bicarbonate | 0.005-1.50 |
| NPK (19:19:19) with trace elements | 0.001-0.10 |
| Magnesium sulphate | 0.003-1.20 |
| Calcium chloride | 0.00016-0.0016 |
| FeSO4 | 0.00009-0.0009 |
| NaCl | 58-117 |

### Example 2

Culturing Dunaliella salina SLS1 in in a Conventional Saline Medium Devoid of Osmotically Active Substance:

The conventional saline medium comprising of components as depicted in Table 2, and atleast 100 gm/l of NaCl prepared in the potable water was used to culture D. salina SLS1. 500 ml of inoculum of D. salina SLS1 as obtained in Example 1 was added to 9.5 litre of the culture medium prepared. The pH was in the range of 6.8-10.4, temperature in the ranges of 24-32° C. and a light intensity in the range of 10-100 Klux. Fresh input of mineral nutrients as depicted in Table 2 may be provided for period of 10 days. The alga was harvested by centrifugation at 12000 rpm for 15 minutes and dewatered by alcoholic treatments.

TABLE 2

| Mineral salts | Concentration g/l | |
|---|---|---|
| Sodium bicarbonate | 0.101 | |
| NPK (19:19:19) with trace elements | 0.01 | |
| Magnesium sulphate | 0.065 | |
| Calcium chloride | 0.0014 | |
| FeSO4 | 0.0009 | |
| NaCl | 100 | g/l |

The harvested Dunaliella salina SLS1 obtained in a conventional saline medium devoid of osmotically active substance showed the biochemical profile as mentioned in Table 3

TABLE 3

| | Dunaliella salina | |
|---|---|---|
| Yield & Biochemical | SLS1 cultured as | |
| profile | per Example 2 | |
| Beta Carotene | 8.6 | |
| Cis Beta Carotene | 1.2 | |
| Alpha carotene | 0.52 | |
| Dry Biomass yield | 66 | mg/l/day |

### Example 3

Culturing Dunaliella salina SLS1 in Medium Containing Sugar Alcohol as Osmotically Active Substance:

The medium comprising of components as depicted in Table 4, and atleast 100 gm/l of glycerol prepared in the potable water was used to culture D. salina SLS1. 500 ml of inoculum of D. salina SLS1 as obtained in Example 1 was added to 9.5 litre of the culture medium prepared. The pH was in the range of 5.8-10.8, temperature in the ranges of 24-32° C. and a light intensity in the range of 10-100 Klux. Fresh input of mineral nutrients as depicted in Table 4 may be provided intermittently for period of 10 days so to increase yield of algal biomass and enrich natural mixed carotenoids. The alga was harvested by centrifugation at 12000 rpm for 15 minutes and dewatered by alcoholic treatments.

TABLE 4

| Mineral salts | Concentration g/l | |
|---|---|---|
| Sodium bicarbonate | 0.101 | |
| NPK (19:19:19) with trace elements | 0.01 | |
| Magnesium sulphate | 0.065 | |
| Calcium chloride | 0.0014 | |
| FeSO4 | 0.0009 | |
| NaCl | 1 | |
| Blue green algae extract | 0.1 | ml/l |

The harvested Dunaliella salina SLS1 showed biochemical profile as given in Table 5 in terms of high carotene content and growth yield as compared to the conventional strain of Dunaliella salina.

TABLE 5

| | Dunaliella salina | | Dunaliella salina | |
|---|---|---|---|---|
| Yield & Biochemical | SLS1 cultured as | | SLS1 cultured as | |
| profile | per Conventional process | | per Example 3 | |
| Beta Carotene | 8.6 | | 14.6 | |
| Cis Beta Carotene | 1.2 | | 1.5 | |
| Alpha carotene | 0.52 | | 0.84 | |
| Dry Biomass yield | 66 | mg/l/day | 96 | mg/l/day |

### Example 4

Culturing of Dunaliella salina SLS1 in Medium Containing Sugar as Osmotically Active Substance.

The medium comprising of components as depicted in Table 6, and atleast 200 gm/l of sucrose prepared in the potable water was used to culture D. salina SLS1. 500 ml of inoculum of D. salina SLS1 as obtained in Example 1 was added to 9.5 litre of the culture medium prepared. The pH was in the range of 6.8-10.4, temperature in the ranges of 24-32° C. and a light intensity in the range of 10-100 Klux. Fresh input of mineral nutrients as depicted in Table 6 may be provided intermittently for period of 10 days so to increase yield of algal biomass and enrich natural mixed carotenoids. The alga was harvested by centrifugation at 12000 rpm for 15 minutes and dewatered by alcoholic treatments.

TABLE 6

| Mineral salts | Concentration g/l | |
|---|---|---|
| Sodium bicarbonate | 0101 | |
| NPK (19:19:19) with trace elements | 0.01 | |
| Magnesium sulphate | 0.065 | |
| Calcium chloride | 0.0014 | |
| FeSO4 | 0.0009 | |
| NaCl | 1 | g/l |

The harvested Dunaliella salina SLS1 showed high biochemical profile as given in Table 7 in terms of carotene content and growth yield as compared to the conventional strain of Dunaliella salina.

TABLE 7

| | Dunaliella salina | | Dunaliella salina | |
|---|---|---|---|---|
| Yield & Biochemical | SLS1 cultured as | | SLS1 cultured as | |
| profile | per Conventional process | | per Example 4 | |
| Beta Carotene | 8.6 | | 13.2 | |
| Cis Beta Carotene | 1.2 | | 1.8 | |
| Alpha carotene | 0.52 | | 0.86 | |
| Dry Biomass yield | 66 | mg/l/day | 87 | mg/l/day |

### Example 5

Culturing of Dunaliella salina SLS1 in Medium Containing Neera as Osmotically Active Substance:

The medium comprising of components as depicted in Table 8, and 9.5 litre double strength Neera was used to culture D. salina SLS1. 500 ml of inoculum of D. salina SLS1 as obtained in Example 1 was added to 9.5 litre of the culture medium prepared. The pH was in the range of 6.4-10.6, temperature in the ranges of 24-32° C. and a light intensity in the range of 10-100 Klux. Fresh input of mineral nutrients as depicted in Table 8 may be provided intermittently for period of 10 days so to increase yield of algal biomass and enrich natural mixed carotenoids. The alga was harvested by centrifugation at 12000 rpm for 15 minutes and dewatered by alcoholic treatments.

TABLE 8

| Mineral salts | Concentration g/l | |
|---|---|---|
| Sodium bicarbonate | 0.101 | |
| NPK (19:19:19) with trace elements | 0.01 | |
| Magnesium sulphate | 0.065 | |
| Calcium chloride | 0.0014 | |
| FeSO4 | 0.0009 | |
| NaCl | 1 | g/l |

The harvested Dunaliella salina SLS1 showed high biochemical profile as given in Table 9 in terms of carotene content and growth yield as compared to the conventional strain of Dunaliella salina.

TABLE 9

| | Dunaliella salina | | Dunaliella salina | |
|---|---|---|---|---|
| Yield & Biochemical | SLS1 cultured as | | SLS1 cultured as | |
| profile | per Conventional process | | per Example 5 | |
| Beta Carotene | 8.6 | | 12.3 | |
| Cis Beta Carotene | 1.2 | | 1.35 | |
| Alpha carotene | 0.52 | | 0.68 | |
| Dry Biomass yield | 66 | mg/l/day | 80 | mg/l/day |

1. A salinity free media for culturing and producing algal biomass comprising at least one osmotically active substance.

2. The salinity free medium as claimed in claim 1, wherein the algae is halophilic algae.

3. The salinity free medium as claimed in claim 2, wherein the halophilic algae is selected from the genus Dunaliella.

4. The salinity free medium as claimed in claim 3, wherein the algae Dunaliella is selected from Dunaliella bardawil and Dunaliella.

5. The salinity free medium as claimed in claim 4, wherein the algae Dunaliella is Dunaliella salina.

6. The salinity free medium as claimed in claim 5, wherein the algae Dunaliella salina is Dunaliella salina SLSI.

7. The salinity free medium as claimed in claim 1, wherein the osmotically active substance is selected from sugar alcohol, sugar, sucralose, palm sugary sap or combinations thereof.

8. The salinity free medium as claimed in claim 7, wherein the concentration of sugar alcohol is from about 30 g/l to about 350 g/l.

9. The salinity free medium as claimed in claim 8, wherein the sugar alcohol is glycerol.

10. The salinity free medium as claimed in claim 7, wherein the concentration of sugar is from about 40 g/l to about 450 g/l.

11. The salinity free medium as claimed in claim 10, wherein the sugar is sucrose.

12. The salinity free medium as claimed in claim 7, wherein the concentration of the palm sugary sap is at least double strength.

13. The salinity free medium as claimed in claim , wherein the concentration of the palm sugary sap is not more than triple strength.

14. The salinity free medium as claimed in claim 7, wherein the palm sugary sap is obtained from palm tree, preferably palmyra palm.

15. The salinity free medium as claimed in claim 7, wherein the concentration of the sucralose is from about 40 g/l to about 450 g/l.

16. The salinity free medium as claimed in claim 1, wherein the medium further comprises minerals and at least 0.1% of sodium chloride.

17. The salinity free medium as claimed in claim 16, wherein the minerals are selected from carbonates or bicarbonates, nitrates, phosphates, sulphates, chlorides, magnesium, calcium, iron, boron, NPK complex, trace elements and/or combination thereof

18. The salinity free medium as claimed in claim 16, wherein the concentration of sodium chloride is from about 1 g/l to about 2 g/l.

19. The salinity free medium as claimed in claim 1, wherein the medium further comprises vitamins, extract from blue green algae and/or combinations thereof.

20. A process for culturing and producing Dunaliella biomass rich in natural mixed carotenoid content comprising;
culturing Dunaliella in a salinity free media for culturing and producing algal biomass comprising at least one osmotically active substance;
allowing the culture to grow under suitable cultural conditions to obtain Dunaliella biomass rich in natural mixed carotenoid content; and
harvesting the Dunaliella biomass.

21. The process as claimed in claim 20, wherein the Dunaliella is selected from Dunaliella bardawil and Dunaliella sauna.

22. The process as claimed in claim 21, wherein the algae Dunaliella sauna is Dunaliella sauna SLS1.

23. The process as claimed in claim 20, wherein the suitable cultural conditions include pH ranging from about 5.8 to about 10.8, temperature ranging from about −2° C. to about 60° C. and light intensity ranging from about 5 klux to 120 klux.

24. The process as claimed in claim 23, wherein the suitable cultural conditions include pH ranging from about 8.6 to about 9.4, temperature ranging from about 20° C. to about 42° C. and light intensity ranging from about 40 klux to 80 klux.

25. The process as claimed in claim 20, wherein the Dunaliella culture is allowed to grow for a period of at least 10 days.

26. The process as claimed in claim 25, wherein the Dunaliella culture is allowed to grow for a period of 12 days to 14 days.

27. The process as claimed in claim 20, wherein the mixed carotenoid content of the Dunaliella biomass is in the range of 12-20%.

28. The process as claimed in claim 20, wherein the yield of Dunaliella biomass rich in mixed carotenoid content on dry basis ranges from about 80 to 100 mg/l of media/day.

29. A Dunaliella biomass as obtained by the process as claimed in claim 20, wherein the Dunaliella biomass is free from salinity and comprises higher concentration of natural mixed carotenoids.

30. The Dunaliella biomass as claimed in claim 29, wherein the carotenoid content of the Dunaliella biomass is in the range of 12-20%.

31. The Dunaliella biomass as claimed in claim 29, wherein the Dunaliella biomass have a temperature tolerance limit from about −2° C. to about 60° C., and a pH tolerance limit from about 5.8 to 10.8.
